(19) United States
(12) Patent Application Publication
(10) Pub. No.: US 2007/0155689 A1
(43) Pub. Date: Jul. 5, 2007

(54) METHODS OF REGULATING GROWTH AND DEATH OF CANCER CELLS

(75) Inventors: Yusuke Nakamura, Yokohama-shi (JP); Yoichi Furukawa, Kawasaki-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ONCOTHERAPY SCIENCE, INC., Minato-ku (JP)

(21) Appl. No.: 11/626,540

(22) Filed: Jan. 24, 2007

Related U.S. Application Data
(62) Division of application No. 10/490,581, filed on Nov. 1, 2004.

(30) Foreign Application Priority Data
Sep. 21, 2001 (JP) 2001-290248

(51) Int. Cl. A61K 48/00; C12Q 1/68; C12N 15/09

(52) U.S. Cl. 514/44; 435/6; 435/455

(57) ABSTRACT

This invention demonstrates that the PEG10 protein suppresses apoptosis and promotes cancer cell growth. Furthermore, PEG10 protein was found to be degraded through interaction with SIAH1 protein. This invention provides methods of regulating cell growth and cell death using the PEG10 protein. Furthermore, the present invention provides methods of screening for novel anticancer agents which use the PEG10 protein. The screening of this invention enables development of pharmaceutical agents that induce apoptosis and suppress cell growth by specifically targeting cancer cells. In particular, since the PEG10 protein is specifically expressed in hepatoma cells, it is an ideal target molecule for diagnosis and treatment of hepatoma.

### CROSS-REFERENCES TO RELATED APPLICATIONS

The present patent application is a divisional application of U.S. patent application No. 10/490,581, filed Nov. 1, 2004, which is a National Stage Entry of PCT/JP02/08416, filed Aug. 21, 2002, which claims the benefit of JP 2001-290248, filed Sep. 21, 2001, the disclosures of each of which are hereby incorporated herein by reference in their entirety for all purposes.

### FIELD OF THE INVENTION

The present invention relates to methods of regulating cell growth by using PEG10, and methods of screening for cancer inhibitors using PEG10.

### BACKGROUND OF THE INVENTION

Primary hepatocellular carcinoma (HCC) is one of the most common malignancies worldwide. Although several modes of novel therapy have been developed over recent years, the prognosis for advanced HCC remains poor. Molecular analysis has revealed that whilst genetic alterations of TP53, CTNNB1 or AXINI are involved in hepatocarcinogenesis (Tanaka, S. et al., Cancer Res. 53, 2884-2887 (1993); and Miyoshi, Y. et al., Cancer Res. 58, 2524-2527 (1998)), they are only involved in a limited fraction of HCCs. Thus the discovery of new target molecules, specifically expressed in tumors and critically involved in a majority of cases, is essential for improving therapeutic intervention and prognosis in hepatic cancers.

Microarray technology has made it possible to obtain comprehensive gene expression data for not only experimental models but also for human cancers (Clark, E. A. et al., Nature 406, 532-535 (2000); and Perou, C. M. et al., Nature 406, 747-752 (2000)). Microarray methods can identify genes whose expression is altered in physiological reactions, in response to various conditions or drugs, or in pathological conditions (Clark, E. A. et al., Nature 406, 532-535 (2000); and Perou, C. M. et al., Nature 406, 747-752 (2000)). Microarray technology can also provide a systematic expression profile of a cell population subset, which may reflect physiological or pathological phenotypes. In a previous report, the present inventors used a 23,040 gene cDNA microarray to compare the expression profiles of 20 HCCs with those of corresponding non-cancerous liver tissue (Okabe, H. et al., Cancer Res. 61, 2129-2137 (2001)). These experiments disclosed a number of genes that may be involved in the development and progression of hepatic cancer, and also revealed that expression profiles differed between HBV- and HCV-positive HCCs (Okabe, H. et al., Cancer Res. 61, 2129-2137 (2001)). Chronic hepatitis due to the hepatitis-B or hepatitis-C virus is considered a major risk factor for HCCs (Stuver, S. O., Semin. Cancer Biol. 8, 299-306 (1998)), but each virus may contribute to the development and progression of hepatic cancer via a different pathway (Koike, K. et al., Hepatology 19, 810-819 (1994); Moriya, K. et al., Nature Med. 4, 1065-1067 (1998)). The difference in expression patterns found by the present inventors suggests a difference in character, although many genes are commonly up-regulated in both types of tumors.

Konopitzky, R. et al. reported R11 as a cancer antigen derived from pancreatic cancer cells (International Publication No. WO01/11040). However, it is unknown whether this protein actually causes malignant transformation or aberrant cell growth, or whether the protein is merely a result of these abnormalities. Neither the activity nor function of the R11 protein is known. Furthermore, expression of R11 in hepatoma cells is completely unknown.

### BRIEF SUMMARY OF THE INVENTION

The present invention provides methods of regulating cell growth by using PEG10. The present invention also provides methods of screening for cancer inhibitors using PEG10.

To discover novel targets for the development of drugs for treating hepatocellular carcinomas (HCCs), the present inventors analyzed the expression profiles of HCCs obtained using a genome-wide cDNA microarray (Okabe, H. et al., Cancer Res. 61, 2129-2137 (2001)), and searched for genes whose expression was commonly and exclusively up-regulated in HCCs. As a result, the present inventors successfully isolated the entire transcript of an EST selectively over-expressed in cancerous tissues but not normal hepatocytes. This gene was eventually found to be identical to paternally expressed gene 10 (PEG10) (Ono, R. et al., Genomics 73, 232-237 (2001)). Immunohistochemical analysis using anti-PEG10 antibody supported the cancer-specific expression of PEG10. Furthermore, PEG10 transfer into hepatoma cells with no endogenous expression of this gene resulted in enhanced cell growth. An antisense S-oligonucleotide that suppresses PEG10 expression resulted in remarkable inhibition of transfected hepatoma cell growth. Furthermore, the present inventors demonstrated that the PEG10 protein interacts with SIAH proteins, which play important roles in apoptosis (Roperch, J. P. et al., Proc. Natl. Acad. Sci. USA. 96, 8070-8073 (1999)). Adenovirus-mediated gene transfer of SIAH1 induced hepatoma cell death, but over-expression of PEG10 in these cells then suppressed this cell death. These findings suggest that suppression of PEG10 activity should be a novel approach to developing drugs for the treatment of hepatocellular carcinomas.

In this way, the present inventors demonstrated for the first time that PEG10 protein comprises the activity of suppressing the death of cancer cells, and of promoting their growth. Furthermore, the present inventors proved that PEG10 protein is regulated by its interaction with SIAH proteins. These findings show that regulating PEG10 protein level, or the interaction between PEG10 protein and SIAH proteins, enables the regulation of cell death and cell growth. Thus, PEG10 protein may serve as an ideal molecular target for the prevention or treatment of cancer. The present invention relates to methods of regulating cell death and cell growth by using PEG10 protein, and methods of screening for cancer inhibitors using PEG10 protein. More specifically, the present invention relates to:

(1) a method of promoting or suppressing cell growth, wherein the method comprises the step of increasing or decreasing PEG10 protein level in a cell, respectively;

(2) a method of suppressing or promoting cell death, wherein the method comprises the step of increasing or decreasing PEG10 protein level in a cell, respectively;

(3) the method of (1) or (2), wherein the step of increasing or decreasing PEG10 protein level in a cell is the step of suppressing or promoting the interaction level of the PEG10 protein with a SIAH protein in a cell;

(4) the method of any one of (1) to (3), wherein the step of increasing PEG10 protein level in a cell is the step of exogenously expressing the PEG10 gene;

(5) the method of any one of (1) to (3), wherein the step of decreasing PEG10 protein level in a cell is the step of transferring antisense polynucleotides against the PEG10 gene into a cell, or expressing the polynucleotides in a cell;

(6) the method of any one of (1) to (5), wherein the cell is a cancer cell;

(7) the method of (6), wherein the cancer cell is a hepatoma cell;

(8) the method of (7), wherein the hepatoma cell is a hepatocellular carcinoma cell;

(9) a method of screening for compounds comprising the activity of regulating cell growth or cell death, the method comprising the steps of: (a) detecting PEG10 gene expression level in a cell in the presence of a sample comprising a test compound; and (b) selecting compounds comprising the activity of increasing or decreasing the expression level;

(10) a method of screening for compounds comprising the activity of regulating cell growth or cell death, the method comprising the steps of: (a) detecting the interaction between the PEG10 protein and a SIAH protein in the presence of a sample comprising a test compound; and (b) selecting compounds comprising the activity of increasing or decreasing the interaction;

(11) a method of screening for compounds comprising the activity of regulating cell growth or cell death, the method comprising the steps of: (a) culturing cells exogenously expressing the PEG10 protein in the presence of a sample comprising a test compound; and (b) selecting compounds comprising the activity of increasing or decreasing growth or cell death of the cells;

(12) a pharmaceutical composition for preventing or treating cell proliferative diseases, comprising compounds able to be isolated by a method of any one of (9) to (11);

(13) the pharmaceutical composition of (12), comprising an antisense polynucleotide against the PEG10 gene;

(14) a method of testing or diagnosing hepatoma, wherein the method comprises the step of detecting structural changes or aberrant expression of the PEG10 gene and/or SIAH gene;

(15) the method of testing or diagnosis of (14), wherein the hepatoma is a hepatocellular carcinoma;

(16) a reagent for testing or diagnosing hepatoma, comprising a polynucleotide which comprises a portion of the PEG10 gene or SIAH gene nucleotide sequence, or a portion of a complementary strand thereof, or an antibody that binds to the PEG10 protein or SIAH protein; and

(17) the reagent for testing or diagnosing hepatoma of (16), wherein the hepatoma is a hepatocellular carcinoma.

PEG10 is a gene up-regulated in a great majority of HCCs (35 of the 36 HCCs analyzed by cDNA microarray, RT-PCR, and/or immunostaining), and expressed at a very low level in corresponding non-cancerous liver tissues. Thus, PEG10 could potentially serve as a diagnostic marker, and an ideal molecular target for the development of drugs to treat patients with primary HCC.

PEG10 transfer to hepatoma cells not expressing detectable endogenous PEG10 protein elicited significant growth-promotion activity. Serum starvation did not suppress the growth of cells that expressed PEG10 at high levels. Since PEG10 transfer into HEK293, Cos7 and NIH3T3 cells did not promote growth (data not shown), the present invention's data indicate that PEG10's oncogenic activity is likely to be hepatocyte-specific.

PEG10 shows 61.4% homology to murine myelin expression factor 3 (MyEF-3), whose product is thought to function as a transcriptional factor and to regulate expression of myelin basic proteins during brain development (Steplewski, A. et al., Biochem. Biophys. Res. Commun. 243, 295-301 (1998)). MyEF-3 protein comprises 235 amino acids, with a zinc-finger domain in the C-terminal region. Conservation of this domain, and the fact that PEG10 is mainly located in the nuclei of HCC cells, indicate that PEG10 itself may function as a transcriptional factor. As the PEG10 N-terminal region also contains a coiled-coil motif, generally facilitating protein-protein interactions, the present inventors used a yeast two-hybrid system to look for proteins that would interact with PEG10. As a result, the present inventors found that PEG10 protein was able to interact with SIAH-1 and SIAH-2 proteins. These SIAH proteins are homologs of Drosophila seven in absentia (sina) gene. SIAH-2 protein is involved in the fate of Drosophila R7 photoreceptor cells during eye development (Carthew, R. W. & Rubin, G. M., Cell 63, 561-577 (1990)). Others have reported that expression of human SIAH1 was induced during apoptosis and p53-dependent cell cycle arrest (Roperch, J. P. et al., Proc. Natl. Acad. Sci. USA. 96, 8070-8073 (1999); and Matsuzawa, S. et al., EMBO J. 17, 2736-2747 (1998)), and that this gene is located on chromosomal band 16q12-q13. This region is frequently deleted in tumors arising from various tissues, including HCCs (Medhioub, M. et al., Int. J. Cancer 87, 794-797 (2000); and Okabe, H. et al., Hepatology 31, 1073-1079 (2000)). These suggested, the possibility that SIAH1 functions as a tumor suppressor. Reduced SIAH1 expression in HCC cell lines, and apoptosis induction after transfer of exogenous SIAH1 into HCC cells, suggest that SIAH1 plays an important role in suppressing hepatocarcinogenesis.

The RING finger domain of SIAH-1 protein is involved in the ubiquitin-mediated proteolysis of several proteins, including kinesin-like DNA binding protein (Kid), BAG-1, and deleted in colorectal cancer (DCC) (Hu, G. et al., Genes Dev. 11, 2701-2714 (1997); Germani, A. et al., Oncogene 19, 5997-6006 (2000); and Hu, G. & Fearon, E. R., Mol. Cell Biol. 19, 724-732 (1999)). SIAH 1 was shown to interact with the tumor suppressor APC, and facilitates degradation of β-catenin through the formation of a degradation complex that is independent of glycogen synthase kinase 3β (GSK3β) (Matsuzawa, S. & Reed, J. C., Mol. Cell 7, 915-926 (2001); and Liu, J. et al., Mol. Cell 7, 927-936 (2001)). The present invention's data demonstrate that SIAH-1 also reduced expression of PEG10 protein in a dose-dependent manner. Therefore, PEG10 is likely to be a target for ubiquitination by SIAH-1. Furthermore, in experiments of the present invention, cell death in cells stably expressing PEG10 was significantly reduced in response to the recombinant adenovirus Ad-SIAH1, which expresses SIAH1. Therefore, an imbalance in the expression of PEG10 and SIAH1 may be involved in hepatocarcinogenesis through apoptosis inhibition. PEG10 was isolated as a paternally expressed gene from a newly defined imprinted region at 7q21 (Ono, R. et al., Genomics 73, 232-237 (2001)). Loss of imprinting might be involved in the elevated expression of this gene in HCC, although no evidence to support this was found (data not shown). Further studies on the PEG10 promoter region will be very helpful in clarifying the mechanism(s) of PEG10 gene up-regulation.

The present inventors demonstrated that reduction of PEG10 expression by treatment with antisense S-oligonucleotides significantly decreases the growth of HCC cells. Interestingly, the antisense sequences only suppressed the growth of HCC cells that endogenously expressed PEG10, and did not suppress growth in cell lines that did not. Expression of this gene was enhanced in the majority of HCC tissues, and very low or absent in all normal adult human tissues except for the gonadal glands. Therefore, PEG10 might be an ideal molecular target in therapeutic strategies for primary HCC. Thus the present invention will contribute to a more profound understanding of hepatocarcinogenesis, and to the development of novel therapeutic approaches.

The present invention provides methods of regulating cell growth and cell death by using PEG10. Methods of regulating cell growth according to this invention comprise the step of increasing or decreasing PEG10 protein level in cells. Increasing PEG10 protein level in cells promotes cell growth, whilst decreasing PEG10 protein level suppresses cell growth. Furthermore, the step of increasing or decreasing PEG10 protein level in cells allows the regulation of cell death. Specifically, increasing PEG10 protein level in cells suppresses cell death. In reverse, decreasing PEG10 protein level promotes cell death. PEG10 protein level can be increased and decreased using various means well known to those skilled in the art.

For example, PEG10 protein can be expressed exogenously in cells. Specifically, for example, the step of transfecting an expression vector comprising a gene encoding the PEG10 protein into a cell enables an easy and effective increase in PEG10 protein level. More specifically, the present invention provides methods for promoting cell growth, and methods for suppressing cell death, wherein these methods comprise the step of inducing the exogenous expression of PEG10 gene. Expression vectors are not particularly limited, and known vector systems including plasmid vectors and viral vectors can be used. Examples of viral vectors that can be used for purposes such as gene therapy include retrovirus vectors, adenovirus vectors, adeno-associated virus vectors, vaccinia virus vectors, lentivirus vectors, herpes virus vectors, alphavirus vectors, EB virus vectors, papillomavirus vectors, and foamy virus vectors. PEG10 gene can also be transfected and expressed using non-viral vectors such as cationic liposomes, ligand DNA complexes, or a gene gun (Niitsu, Y. et al., Molecular Medicine 35: 1385-1395 (1998)). The gene may be transferred to cultured cells, tissues, organs, or to a living body. Gene transfer into a living body may be performed in vivo or ex vivo.

PEG10 protein level in cells can be increased by transfecting cells with PEG10 protein prepared in vitro. This can be accomplished, for example, by transferring into cells a recombinant PEG10 protein combined with a known transfection reagent. Alternatively, PEG10 protein can be injected into cells using a microinjector.

PEG10 protein level can be decreased by inhibiting PEG10 gene expression, or by promoting PEG10 protein degradation. The present inventors demonstrated that PEG10 protein expression is decreased by SIAH1 protein, which is involved in ubiquitin-proteosome-mediated proteolysis. Therefore, for example, an increase in the level of SIAH1 protein in cells, or the promotion of PEG10 protein ubiquitination, can facilitate PEG10 protein degradation and thus decrease the PEG10 protein level in cells. To increase the level of SIAH1 protein in cells, a method similar to the above-mentioned method for increasing PEG10 protein level can be applied to SIAH1. For example, SIAH1 protein level may be increased by transfecting cells with viral vectors carrying the SIAH1 gene in order to express exogenous SIAH1 protein.

The suppression of PEG10 protein degradation in cells allows an increase in PEG10 protein level. For example, decreasing the level of SIAH1 protein in cells will suppress PEG10 protein degradation by SIAH1 protein, and PEG10 protein level in the cells will increase. SIAH1 protein level can be decreased, for example, by inhibiting SIAH1 gene expression, or by promoting SIAH1 protein degradation. Furthermore, SIAH protein activity may be inhibited by SIAH protein inhibitors.

PEG10 gene expression can be inhibited by antisense polynucleotides of PEG10 gene. The term “PEG10 gene” refers to polynucleotides encoding PEG10 protein. PEG10 gene may have a naturally occurring or artificially prepared nucleotide sequence. As long as it encodes PEG10 protein, PEG10 gene in this invention includes, for example, polynucleotides comprising optional nucleotide sequences constructed based on codon degeneracy. PEG10 gene may be derived from mRNA, cDNA, or genomic DNA. PEG10 gene may include untranslated sequences (UTS), and those comprising introns, and these UTS and introns are also included as parts of the PEG10 gene in this invention. Antisense polynucleotides against the PEG10 gene include polynucleotides that hybridize to any part of a PEG10 gene transcript. PEG10 gene transcripts include not only spliced mature mRNA, but also early transcripts that have not been spliced, and intermediates formed in the splicing process. For example, in the present invention, antisense polynucleotides include polynucleotides complementary to nucleotide sequences of the UTS of PEG10 gene transcripts, exons, or introns. The present invention demonstrated that antisense polynucleotides encompassing the first exon-intron boundary of PEG10 gene significantly decrease PEG10 gene expression. Thus, antisense polynucleotides that cover exon-intron or intron-exon boundaries may be effective in inhibiting PEG10 gene splicing. Alternatively, they may be antisense polynucleotides against regions containing translation initiation codons on PEG10 transcripts.

The antisense polynucleotide may be an antisense polynucleotide that covers the entire coding region of PEG10 protein, or an antisense polynucleotide against a small portion of a PEG10 gene transcript. As long as PEG10 protein expression can be effectively inhibited, the antisense polynucleotide does not have to be completely complementary to a PEG10 gene nucleotide sequence or a partial sequence thereof. However, it preferably has 70% or higher, more preferably 80% or higher, or even more preferably 90% or higher identity to the nucleotide sequence of the PEG10 gene antisense strand. More preferably, it has complete (100%) identity. Nucleotide sequence identity can be determined, for example, by using Karlin and Altschul's BLAST algorithm (Karlin, S. and Altschul, S. F., Proc. Natl. Acad. Sci. USA 87:2264-2268, 1990; and Proc. Natl. Acad. Sci. USA 90:5873-5877, 1993). When using the BLAST and Gapped BLAST programs, respective default parameters are used. Specific techniques for these analysis methods are well known (see BLAST (Basic Local Alignment Search Tool) at the NCBI (National Center for Biotechnology Information) website: http://www.ncbi.nlm.nih.gov). Alternatively, an antisense polynucleotide against PEG10 gene is preferably a polynucleotide comprising a nucleotide sequence completely complementary to at least 15 or more, preferably 16 or more, and more preferably 17, 18, 20 or more continuous nucleotides in the nucleotide sequence of the PEG10 gene mRNA (that is, with 100% identity to the antisense strand nucleotide sequence). Antisense polynucleotides may be either DNA or RNA, and may also comprise modified nucleotides. Furthermore, they may be nucleic acid analogues such as peptide nucleic acids (PNAs). Antisense polynucleotides against PEG10 gene are useful as cell growth inhibitors, and cell death accelerators (apoptosis accelerators). Particularly, antisense polynucleotides against PEG10 gene are useful as agents (including reagents and drugs) for inducing cancer cell death and suppressing cancer cell growth, particularly for hepatoma cells, and more preferably for hepatocellular carcinoma cells.

Antisense polynucleotides can inhibit PEG10 gene expression when they are transferred into or expressed in cells. For example, in order to express antisense polynucleotides in cells, the cells are transfected with expression vectors encoding the antisense polynucleotides. As described above, expression vectors may be viral vectors such as retrovirus vectors, adenovirus vectors, and adeno-associated virus vectors; and non-viral vectors such as liposomes. Furthermore, by directly incorporating antisense polynucleotides into cells, PEG10 gene expression can be inhibited. For example, cells may be transfected with antisense oligonucleotides comprising about 10 to about 30 nucleotides. The present inventors successfully suppressed PEG10 gene expression by transfecting cells with antisense oligonucleotides against PEG10. Therefore, antisense oligonucleotides against PEG10 are preferably used for suppressing PEG10 gene expression. Antisense polynucleotides may be composed of natural nucleotides, or may include modified nucleotides. Phosphorothioate oligonucleotides (S-oligos) are particularly suitable as antisense polynucleotides of this invention. Phosphorothioate oligonucleotides include those in which phosphorothioates are partially incorporated (partial thio-modification).

In the present invention, “PEG10” protein includes human PEG10, as identified by GenBank Accession No. NM_{—}015068 (Ono, R. et al., Genomics 73,232-237(2001)), and also includes structurally similar proteins, such as homologs and derivatives comprising functions equivalent to human PEG10. “Comprising functions equivalent to human PEG10” means comprising the activity of promoting cell growth or suppressing cell death. The activity is significant compared to when the protein of interest is not expressed. For example, activity judged using statistical methods to have a significance level of 5% or more is deemed significant. For example, these activities can be measured by transfecting the desired cell with an expression vector encoding the protein of interest, and then determining cell growth or cell death. Details of these measurement methods are described later. In particular, hepatocytes or cells derived from hepatocytes (including hepatocellular carcinoma cells) are suitable cells. For example, cells that do not express or hardly express endogenous PEG10 protein may be used. For example, measurements can be made using SNU423, SNU449, SNU475, or such, available from the Korean Cell Line Bank. Homologs of human PEG10 protein include counterparts possessed by other organisms, which comprise functions equivalent to human PEG10 protein. Furthermore, derivatives include proteins comprising the amino acid sequence of human PEG10 protein or a homolog thereof, in which one or more amino acids have been substituted, deleted, added, and/or inserted. Derivatives are not limited to naturally existing proteins, and may be prepared by methods for artificially modifying amino acid sequences. This artificial modification can be carried out, for example by performing site-directed mutagenesis on DNA that encodes the protein using techniques well known to those skilled in the art, that is, using deletion-mutant construction, PCR method, cassette mutation method, or the like. The number of amino acids to be modified is not limited as long as a function equivalent to human PEG10 protein is present, but typically is not more than 10%, preferably not more than 5%, and more preferably not more than 1% of all amino acids of the original human PEG10 protein or homolog thereof. More specifically, the number of amino acids to be modified is preferably 30 or less, more preferably 20 or less, even more preferably ten or less, and even more preferably five or less. It is preferable that amino acid sequence modification does not occur for amino acids essential to activity in the PEG10 protein C-terminal region zinc finger domain, and the N-terminal region coiled-coil motif, and amino acids generally conserved in these motifs. Amino acids conserved in these motifs are well known to those skilled in the art. In the present invention, structurally similar proteins, such as homologs and derivatives of human PEG10 protein, are preferably highly homologous to the amino acid sequence of human PEG10 protein. Highly homologous means having a sequence identity of at least 70% or more, preferably 80% or more, more preferably 90% or more, even more preferably 95% or more, and still more preferably 99% or more. Amino acid sequence identity can be determined, for example, using Karlin and Altschul's BLAST algorithm (Karlin, S. and Altschul, S. F., Proc. Natl. Acad. Sci. USA 87:2264-2268, 1990; and Proc. Natl. Acad. Sci. USA 90:5873-5877, 1993). Based on this algorithm, a program called BLASTX has been developed (Altschul et al., J. Mol. Biol. 215:403-410, 1990). When amino acid sequences are analyzed using BLASTX, parameters are set, for example, as follows: score=50; and wordlength=3. When using BLAST and Gapped BLAST programs, respective default parameters are used. Specific techniques for these analysis methods are well known (see Basic Local Alignment Search Tool (BLAST) at the National Center for Biotechnology Information (NCBI) website).

In the present invention, “SIAH proteins” include human SIAH1 and SIAH2 proteins known to be human homologs of Drosophila seven in absentia gene; and proteins structurally similar to human SIAH1 or SIAH2 proteins, such as homologs and derivatives comprising functions equivalent to these proteins. Human SIAH1 is as indicated in GenBank Accession Nos. NM_{—}003031, XM_{—}008013, U76247, and such (Nemani, M. et al., Proc. Natl. Acad. Sci. USA. 93, 9039-9042 (1996); Hu, G. et al., Genes Dev. 11, 2701-2714 (1997); and Hu, G. et al., Genomics 46, 103-111 (1997)). Furthermore, human SIAH2 is as indicated in GenBank Accession Nos. NM_{—}005067, XM_{—}003089, XM_{—}042171, Y15268, U76248, and such (Hu, G. et al., Genes Dev. 11, 2701-2714 (1997); Hu, G. et al., Genomics 46, 103-111 (1997); and Germani, A. et al., Mol. Cell. Biol. 19, 3798-3807 (1999)). A SIAH protein in the present invention is preferably a human SIAH1 protein, or a protein structurally similar to a human SIAH1 protein, including SIAH1 homologs and derivatives comprising functions equivalent to this protein.

“Comprising functions equivalent to human SIAH1 protein” means interacting with any one of the PEG10 proteins of this invention to suppress cell growth-promoting activity or cell death-suppressing activity due to the PEG10 protein. Such an activity is significant compared to when the SIAH protein of interest is not expressed. For example, using statistical methods, activities are judged to be significant at a significance level of 5% or more. For example, these activities can be measured by transfecting cells that express PEG10 protein with an expression vector encoding the protein of interest, and determining cell growth or cell death. Details of these assays are described later. Cells that may be used include cancer cells whose endogenous PEG10 protein expression is enhanced, and cells made to exogenously express PEG10 protein. In particular, hepatocytes or cells derived from hepatocytes (including hepatocellular carcinoma cells) are suitable cells. For example, suitable cells include those from hepatocellular carcinoma cell lines with enhanced PEG10 protein expression, such as HepG2, Huh7, or Alexander. Alternatively, SNU423, SNU449, or SNU475 cells, which do not express endogenous PEG10 protein but have been induced to express PEG10 protein exogenously, may be used. Human SIAH1 or SIAH2 protein homologs include counterparts possessed by other organisms and comprising a function equivalent to SIAH1 or SIAH2 protein. For example, mouse proteins, Xenopus proteins, and such are already known human SIAH1 and SIAH2 homologs (see NM_{—}009172, Z19580, M38384, Z19581, U89792, AF155509) (Della, N. G. et al., Development 117, 1333-1343 (1993); and Bogdan, S. et al., Mech. Dev. 103, 61-69 (2001)). Derivatives also include proteins comprising the amino acid sequence of human SIAH1 or SIAH2 protein, or homologs thereof, in which one or more amino acids has been substituted, deleted, added, and/or inserted. Derivatives include naturally existing proteins, and proteins whose amino acid sequences have been artificially modified. The number of amino acids to be modified is not limited as long as a function equivalent to human SIAH1 or SIAH2 protein is present. The number of amino acids to be modified is expected to typically be not more than 10%, preferably not more than 5%, and more preferably not more than 1% of the total amino acids of the original human SIAH1 or SIAH2 protein, or homolog thereof. More specifically, the number of amino acids to be modified is preferably 30 or less, more preferably 20 or less, even more preferably ten or less, and still more preferably five or less. In the present invention, structurally similar proteins such as homologs and derivatives of human SIAH1 or SIAH2 protein are preferably highly homologous to the amino acid sequence of human SIAH1 or SIAH2 protein. Highly homologous refers to sequence identify of at least 70% or more, preferably 80% or more, more preferably 90% or more, even more preferably 95% or more, and still more preferably 99% or more. Amino acid sequence identity can be determined as described above.

In order to isolate cDNAs which encode structurally similar proteins, such as homologs of a given protein, a cDNA library can be screened using the cDNA of that protein as a probe. One skilled in the art can properly select stringent hybridization conditions for obtaining DNAs that encode proteins which are structurally similar and functionally equivalent to a protein such as a PEG10 or SIAH protein homolog. As an example, in a hybridization solution comprising 25% formamide, more stringently 50% formamide, and 4×SSC, 50 mM Hepes (pH 7.0), 10×Denhardt's solution, and 20 μg/mL denatured salmon sperm DNA, pre-hybridization is performed overnight at 42° C., a labeled probe is added, and hybridization is performed by incubating overnight at 42° C. A subsequent wash can be performed using washing solution and temperature conditions of about “1×SSC, 0.1% SDS, 37° C.”, more stringent conditions are about “0.5×SSC, 0.1% SDS, 42° C.”, even more stringent conditions are about “0.5×SSC, 0.1% SDS, 60° C.”, and still more stringent conditions are about “0.2×SSC, 0.1% SDS, 65° C.”. As hybridization washing conditions become more stringent, DNA with higher homology to the probe sequence is expected to be isolated. However, in addition to the above-mentioned combinations of SSC, SDS, and temperature conditions, those skilled in the art can accomplish stringencies such as those mentioned above by properly combining the above-mentioned factors or other factors that determine hybridization stringency (for example, type of carries such as membranes and the like, composition of the hybridization solution, length of the probes, GC content, etc).

PEG10 protein level in cells can be increased or decreased by promoting or suppressing the interaction level of PEG10 protein with SIAH proteins in cells. The present inventors found that PEG10 protein interacts with SIAH proteins, and demonstrated that this interaction regulates PEG10 protein-related cell growth and cell death. The phrase, “interaction level of PEG10 protein with SIAH proteins in cells” refers to the portion (proportion) of PEG10 protein interacting with SIAH protein, relative to total PEG10 protein in the cells. Increasing the cellular level of PEG10 protein interaction with SIAH proteins promotes PEG10 protein degradation, and suppresses cell growth. Conversely, decreasing the level of PEG10 protein interaction with SIAH proteins suppresses PEG10 protein degradation, and promotes cell growth. Equally, increasing the interaction level of PEG10 protein with SIAH proteins in cells promotes PEG10 protein degradation, and promotes cell death. Conversely, decreasing the interaction level of PEG10 protein with SIAH proteins suppresses PEG10 protein degradation, and suppresses cell death. The interaction level of PEG10 protein with SIAH proteins in cells can be increased, for example, by increasing SIAH protein level in cells. Expressing SIAH proteins in cells with enhanced PEG10 protein expression causes a reduction in PEG10 protein expression level, and induces cell death.

Promotion or suppression of cell growth and cell death can be tested using known methods. For example, and as described in the Examples, cell growth can be detected by assays using tetrazolium salts such as 3-(4,5-dimethylthiazol-2-yl)-2,5-diphenyltetrazolium bromide (MTT) and 2-(4-Iodophenyl)-3-(4-nitrophenyl)-5-(2,4-disulfophenyl)-2H-tetrazolium salt (WST-1); BrdU uptake assays; or cell cycle measurements using flow cytometry. Cell death can be detected, for example, through viable cell measurements using trypan blue exclusion, the above mentioned cell-cycle measurements, or various apoptosis assays. In the present invention, cell death regulation particularly comprises apoptosis regulation. Apoptosis can be detected, for example, by observing nuclear condensation and fragmentation using a microscope, or by using a terminal transferase-mediated dUTP nick end-labeling (TUNEL) assay or DNA ladder detection. These assays can be performed using common techniques well known to those skilled in the art. A TUNEL assay can be performed, for example, by using Apoptag Direct (Oncor), according to the attached instructions.

In the present invention, there are no particular limitations on cells targeted for cell growth or cell death regulation, as long as cell growth or cell death is regulated in response to the regulation of PEG10 protein level. The cells are not particularly limited as long as they express PEG10 protein, however, suitable examples include cancer cells. Specifically, hepatoma cells are preferred in this invention. Hepatomas include hepatocellular carcinoma and cholangiocellular carcinoma. In the present invention, hepatocellular carcinomas (HCC) are particularly suitable hepatoma cells for suppressing cell growth and/or promoting cell death. In the present invention, HCCs particularly include primary hepatocellular carcinomas, and also metastasized carcinomas derived from primary HCCs. By applying the method of this invention to cancer cells and decreasing PEG10 protein level in cancer cells, the growth of these cancer cells can be suppressed, and apoptosis can be induced. PEG10 protein expression is enhanced in primary HCCs, and its expression is hardly observed in normal liver. Therefore, regulating PEG10 protein level may be an ideal strategy for preventing and treating HCCs. Furthermore, applying the method of the present invention to normal cells or non-cancerous cells may also be useful. In the majority of organs, excluding germ cells, PEG10 gene expression is not detectable, or is hardly expressed. By increasing PEG10 protein level in these cells and promoting cell growth, these cells can be used as cancer cell models. Such cells are not expected to comprise mutations other than in the PEG10 gene, and are useful in screening for drugs which target PEG10 protein. Liver cells, specifically hepatocytes, are preferred in the promotion of cell growth or suppression of cell death.

The present invention also provides methods of screening for compounds comprising the activity of regulating cell growth or cell death, as described below. Compounds selected using such screening may be used to regulate the above-mentioned cell growth and cell death. In this invention, one embodiment of a method of screening for compounds comprising the activity of regulating cell growth or cell death comprises the steps of (a) detecting PEG10 gene expression level in a cell in the presence of a sample comprising a test compound, and (b) selecting compounds comprising the activity of increasing or decreasing the expression level. Specifically, this screening method includes methods which comprise the following steps:

(a) detecting PEG10 mRNA in cells in the presence of a sample comprising a test compound, and (b) selecting compounds comprising the activity of increasing or decreasing that mRNA level;

(a) detecting PEG10 protein in cells in the presence of a sample comprising a test compound, and (b) selecting compounds comprising the activity of increasing or decreasing that protein level;

(a) detecting the transcriptional activity of the PEG10 gene promoter in cells in the presence of a sample comprising a test compound, and (b) selecting compounds comprising the activity of increasing or decreasing that transcriptional activity; or (a) detecting PEG10 protein activity in cells in the presence of a sample comprising a test compound, and (b) selecting compounds comprising the activity of increasing or decreasing that activity.

PEG10 mRNA can be detected using known methods such as Northern blotting and RT-PCR. A specific example of RT-PCR analysis is described in Example 3. Expression level can be more accurately determined using TaqMan PCR or the like (Okabe, H. et al., Cancer Res. 61, 2129-2137 (2001)). Furthermore, the PEG10 protein can be detected by Western blotting using anti-PEG10 protein antibodies, or by immunoprecipitation, enzyme linked immunosorbent assay (ELISA), immunohistochemical staining, and such. Antibodies against PEG10 protein can be prepared by known methods using synthetic peptides comprising a full-length or partial amino acid sequence of the PEG10 protein. A PEG10 antibody may be a polyclonal or monoclonal antibody. Specific examples of Western blotting and immunohistochemical staining using PEG10 antibodies are described in Example 3.

Samples comprising a test compound for use in the screening of this invention can include, but are not limited to, libraries of small-molecule synthetic compounds, purified proteins, expression products of gene libraries, libraries of natural or synthetic peptides, cell extracts, cell culture supernatants, natural compounds, and serum components.

Cells for use in such a screening are the same as those described above. For example, and without particular limitation, cells for use when screening for compounds that decrease PEG10 gene expression include cancer cell strains or primary cancer cells with enhanced PEG10 gene expression. Hepatoma cells, especially hepatocellular carcinoma cells (HCCs) are suitable for use. Since PEG10 gene expression is enhanced in most HCC cells, these cells, such as HepG2, Huh7, and Alexander, may be used for screening. These cells are cultured in the presence of a sample comprising a test compound, and PEG10 mRNA or protein expression is measured. The compounds selected are those which increase or decrease PEG10 mRNA or protein level compared to expression level when a similar operation is performed in the absence of a test compound. Compounds that increase PEG10 mRNA or protein level are useful as pharmaceutical agents for promoting PEG10 gene expression, facilitating cell growth, and suppressing apoptosis. Compounds that decrease PEG10 mRNA or protein level are useful as pharmaceutical agents for decreasing PEG10 gene expression, suppressing cell growth, and facilitating apoptosis. In particular, compounds that decrease PEG10 gene expression may be used as preventive or therapeutic agents for cancer.

Screening may also be performed using a PEG10 gene promoter. In this method, screening is performed using cells transfected with a reporter construct prepared by functionally binding a transcription regulatory region which regulates PEG10 gene expression, or a partial sequence thereof, to a reporter gene. Specifically, the method comprises the steps of (a) detecting reporter gene expression level in cells comprising the reporter construct, in the presence of a sample comprising a test compound, and (b) selecting compounds that increase or decrease reporter gene expression level compared to when the test compound is absent. The phrase, “functionally binding” refers to the binding of the reporter gene to the promoter region which regulates PEG10 gene expression, or to a partial sequence thereof, such that regulation of reporter gene expression depends on the sequence of the promoter region, or the partial sequence. Promoter sequences derived from genes other than PEG10 gene may be included upstream of the reporter gene. For example, the smallest unit of a promoter sequence derived from SV40 or another widely used promoter can be included. Additionally, PEG10 gene-derived transcription regulatory sequences may be placed close to the promoter sequence. Assays using such chimeric promoters are commonly performed by those skilled in the art.

The PEG10 gene promoter sequence can be isolated from genomic DNA. For example, DNA of the upstream region of the PEG10 transcription initiation site is isolated by screening a genomic DNA library or the like, using PEG10 cDNA as a probe. In general, a number of regulatory sequences regulating expression are located in the sequence several kb upstream of the transcription initiation site. Transcription regulatory sequences which regulate PEG10 gene expression can be specified, for example, by preparing a series in which the isolated DNA is deleted in a stepwise fashion, using the reporter gene to perform promoter assays. More specifically, decreased transcriptional activity due to site-directed mutagenesis can be confirmed, or the involvement of transcriptional factors can be confirmed, by using oligonucleotides comprising a candidate sequence in an electrophoretic mobility shift assay (EMSA), competition assay, or the like (J. Sambrook and D. W. Russell eds., “Molecular cloning: a laboratory manual”, 3rd ed., Cold Spring Harbor Laboratory Press, Cold Spring Harbor, N.Y., 2001). Reporter genes are not particularly limited as long as their expression is detectable, and any one of the reporter genes generally used by those skilled in the art for various analysis systems, such as the luciferase gene, chloramphenicol acetyl transferase (CAT) gene or β-galactosidase gene, may be used.

Cells used for this screening are the same as those mentioned above. For example, and without particular limitation, cancer cell strains or primary cancer cells with enhanced PEG10 gene expression can be used when screening for compounds that decrease PEG10 gene expression. Hepatoma cells, especially HCC cells, are suitable for use. PEG10 gene expression in HCC is enhanced in most cells, and any one of these cells, such as HepG2, Huh7 or Alexander, can be used for screening. Furthermore, transfer of reporter vectors into these cells can be performed by utilizing various methods well known to those skilled in the art, including pulse electroporation and lipofectin. In addition, expression level detection depending on the type of the reporter gene can be carried out by methods generally performed by those skilled in the art. For example, when the luciferase gene is used as the reporter gene, luciferase activity can be measured using the commercially available Dual Luciferase system (Promega).

Detection results are evaluated by comparison with expression level when a similar operation is performed without the addition of a test compound. As a result, compounds that increase or decrease reporter gene expression level are selected. Compounds that increase reporter gene expression are useful as pharmaceutical agents for promoting PEG10 gene expression, enhancing cell growth, and suppressing apoptosis. Compounds that decrease reporter gene expression are useful as pharmaceutical agents for decreasing PEG10 gene expression, suppressing cell growth, and promoting apoptosis. Compounds that decrease PEG10 gene expression are expected to be used particularly as preventive or therapeutic agents for cancer.

Furthermore, screening can be performed by detecting PEG10 protein activity. The present inventors demonstrated that the PEG10 protein comprises the activity of translocating into the nucleus. The present inventors also elucidated that the PEG10 protein comprises the activity of promoting cell growth, and the activity of suppressing cell death. In addition, the present inventors found that the PEG10 protein comprises the activity of binding to SIAH proteins. The PEG10 protein has a zinc finger domain in the C-terminal region, and is analogous to MyEF-3 protein, which is known as a transcriptional factor. The fact that PEG10 is localized mainly in the nucleus supports the possibility that PEG10 functions as a transcriptional factor. The use of these PEG10 protein activities as indicators enables screening for compounds comprising the activity of regulating cell growth or cell death. For example, screening using nuclear localization as an indicator can be performed specifically by methods comprising the steps of (a) detecting nuclear localization of PEG10 protein in cells in the presence of a sample comprising a test compound, and (b) selecting compounds comprising the activity of promoting or suppressing that nuclear localization.

Nuclear localization of PEG10 protein can be detected by performing immunohistochemical staining using anti-PEG10 antibody, or by preparing nuclei from cells and using immunoprecipitation or Western blotting to detect PEG10 protein contained in the nuclear fractions. Alternatively, intracellular localization of PEG10 protein can be detected more conveniently by cellular expression of a fusion protein, where a peptide label such as GFP is attached to the PEG10 protein. If a sample comprising a test compound causes the proportion of PEG10 protein in the nucleus to increase or decrease, relative to PEG10 protein in the cell, the compound is determined to promote or suppress nuclear localization of PEG10 protein, respectively.

One of the present inventors' important discoveries is that interaction of PEG10 protein with SIAH protein leads to a decrease in the PEG10 protein-induced activities of cell growth-promotion and apoptosis suppression. As described above, the present invention provides methods of screening for compounds comprising the activity of regulating cell growth or cell death, in which a method comprises the steps of (a) detecting the interaction between PEG10 protein and SIAH proteins in the presence of a sample comprising a test compound, and (b) selecting compounds comprising the activity of increasing or decreasing this interaction. Compounds that promote or suppress the interaction between PEG10 protein and SIAH proteins can be used to regulate cell growth and cell death. Interaction between PEG10 protein and SIAH proteins comprises binding between PEG10 protein and SIAH protein, as well as protein modification (including ubiquitinylation), structural change, activation, inactivation, degradation, stabilization or change in intracellular localization, which depend on contact between PEG10 protein and SIAH protein. These interactions can be detected by well known molecular biological techniques.

For example, screening methods using binding between PEG10 protein and a SIAH protein as an indicator comprise the steps of (a) contacting PEG10 protein with an SIAH protein in the presence of a sample comprising a test compound, (b) detecting binding between the PEG10 protein and the SIAH protein, and (c) selecting compounds that promote or suppress binding between the PEG10 protein and the SIAH protein, compared to measurements made in the absence of the sample. Furthermore, the present invention also includes a screening method comprising the steps of (a) contacting complexes comprising the PEG10 protein and a SIAH protein with a sample comprising a test compound, (b) detecting binding between the PEG10 protein and the SIAH protein, and (c) selecting compounds that promote or suppress binding of the PEG10 protein with the SIAH protein, compared to measurements made in the absence of the sample. The PEG10 protein or SIAH protein used in the screening may be a deletion protein such as a partial peptide, or a protein to which other peptides have been added, as long as both proteins bind to each other. Human PEG10 protein or fragments thereof, and human SIAH protein or fragments thereof, are appropriate for use as the PEG10 protein and SIAH protein respectively.

Step (a) can be performed, for example, by adding a sample comprising a test compound, PEG10 protein, and an SIAH protein to the same solution, but is not limited thereto. For example, an extract can be prepared from cells exogenously expressing PEG10 protein and an SIAH protein, and this extract can be suspended in an appropriate buffer, and added to a sample comprising a test compound. Alternatively, a sample comprising a test compound can be added to the culture medium of cells exogenously expressing PEG10 protein and an SIAH protein, and a cell extract can be prepared from this.

The binding detection of step (b) can be performed by one skilled in the art using well known methods such as immunoprecipitation, Western blot analysis, or combinations thereof. For example, binding can be detected by immunoprecipitation using antibodies against PEG10 protein, and antibodies against SIAH protein. Specifically, for example, a complex of PEG10 protein and an SIAH protein is immunoprecipitated from the solution of step (a) using an antibody against the PEG10 protein or SIAH protein. Next, Western blot analysis is performed on the precipitated immunocomplex, using an antibody against the other protein to evaluate whether both proteins have bound.

Additionally, the binding activity of step (b) can be measured using a pull-down assay; ELISA; a two-hybrid method using yeast or mammalian cells (see the Examples); an in vitro binding experiment using recombinant proteins synthesized using E. coli or proteins synthesized by an in vitro protein synthesis system using in vitro translation; immunoprecipitation; and immunostaining. Furthermore, screening may be performed by using BIACORE and such to detect protein-protein interactions using surface plasmon resonance. In addition, the following methods are well known to those skilled in the art: methods of immobilizing a protein molecule of the PEG10 protein-SIAH protein complex, reacting it with a synthetic compound, natural product bank or the like, and then screening for molecules that inhibit binding between the PEG10 protein and SIAH protein; and methods of isolating a compound of interest using high throughput screening according to combinatorial chemistry techniques. Furthermore, screening may be performed through protein-protein interaction analysis, by fluorescence polarization measurements using fluorescent-labeled proteins.

When the measured interaction between PEG10 protein and a SIAH protein is reduced compared to that measured in the absence of a test compound, that test compound becomes a candidate compound for promoting cell growth, and for suppressing cell death. These compounds are useful as cell growth promoters and cell death inhibitors (particularly apoptosis inhibitors). If this interaction increases, the test compound becomes a candidate compound for suppressing cell growth, and for promoting cell death. These compounds are useful as cell growth inhibitors, and cell death promoters (particularly as apoptosis promoters). In particular, compounds which promote the interaction between the two proteins are expected to be used as pharmaceutical agents for preventing or treating cell proliferative diseases including cancer, and diseases accompanying apoptosis disorders.

In particular, the present invention provides cell growth inhibitors and cell death promoters comprising a PEG10 gene antisense polynucleotide. The PEG10 gene antisense polynucleotide may be applied as a pharmaceutical agent against cell proliferative diseases and diseases accompanying apoptosis disorders. The PEG10 gene antisense polynucleotide can be administered to patients, for example, by ex vivo or in vivo methods using viral vectors such as retrovirus vectors, adenovirus vectors and adeno-associated virus vectors, and non-viral vectors such as liposomes. Such gene therapies enable the suppression of cell growth by lifting apoptosis inhibition through the inhibition of PEG10 protein expression in a patient's target cells.

In the present invention, the PEG10 gene antisense polynucleotide includes polynucleotides comprising sequences complementary to the nucleotide sequences of PEG10 gene transcripts, as described above. For example, the antisense polynucleotide of the PEG10 gene may be an antisense polynucleotide comprising a sequence complementary to a PEG10 mRNA UTS, exon, or intron sequence. “Complementary” means having preferably 70% or more, more preferably 80% or more, even more preferably 90% or more identity to the nucleotide sequence of the PEG10 gene antisense strand. More preferably, it has complete (100%) identity. Nucleotide sequence identity can be determined, for example, by using Karlin and Altschul's BLAST algorithm (Karlin, S. and Altschul, S. F., Proc. Natl. Acad. Sci. USA 87:2264-2268, 1990; Proc. Natl. Acad. Sci. USA 90:5873-5877, 1993). When using BLAST and Gapped BLAST programs, respective default parameters are used. Specific techniques for these analysis methods are well known (see BLAST at the NCBI website). The PEG10 gene transcripts include not only spliced mature mRNA, but also early transcripts that have not been spliced, and intermediates formed during the splicing process. A particularly suitable antisense polynucleotide of this invention is an antisense polynucleotide encompassing at least one exon-intron or intron-exon boundary on the PEG10 gene. More preferably, it is an antisense polynucleotide encompassing the first exon-intron boundary of the PEG10 gene. The term “encompassing” means comprising a nucleotide sequence complementary to the nucleotide sequence of the sense strand of a continuous region of at least 15 nucleotides, preferably 16 or more nucleotides, and more preferably 17, 18, 20, or more nucleotides comprising the boundary region. The antisense polynucleotide may also be an antisense polynucleotide encompassing the translation initiation codon of the PEG10 transcript. The antisense polynucleotide may be DNA or RNA, and may also contain modified nucleotides. Phosphorothioate oligonucleotides (S-oligos) are particularly suitable as the antisense polynucleotide of this invention. Phosphorothioate oligonucleotides may include those with partial phosphorothioate incorporation (partial thio-modification). Furthermore, the antisense polynucleotide may also be nucleic acid analogues such as PNA. The present invention provides pharmaceutical agents comprising antisense polynucleotides of the PEG10 gene. These pharmaceutical agents are useful for the prevention and treatment of cell proliferative diseases.

The present invention provides methods for preventing or treating cell proliferative diseases comprising the step of administering a compound or PEG10 gene antisense polynucleotide obtainable using the screening methods of this invention. Furthermore, the present invention provides pharmaceutical compositions for the prevention or treatment of cell proliferative diseases comprising the above-mentioned compounds or antisense polynucleotides. Examples of cell proliferative diseases include cancer, and in particular, hepatoma.

When using the compounds and PEG10 gene antisense polynucleotides which can be isolated using a screening method of this invention as pharmaceutical compositions, the compounds or antisense polynucleotides themselves can be directly administered to patients, or else administered upon formulation using conventional preparation methods. The present invention relates to pharmaceutical compositions comprising a compound, or particularly PEG10 gene antisense polynucleotides, which can be selected by the above-mentioned screening. The pharmaceutical compositions of the present invention are useful for the regulation of cell death and cell growth. For example, they may be administered after formulation by appropriate combination with a pharmaceutically acceptable carrier or vehicle, such as sterilized water, physiological saline, vegetable oil, emulsifier, suspending agent, surfactant, stabilizer, controlled-release agent and the like. When used as a pharmaceutical composition, they may be in the form of an aqueous solution, tablet, capsule, troche, buccal preparation, elixir, suspension, syrup, nasal drop, inhalant solution, or such. The relative content of compounds in the compositions may be appropriately determined. Administration to patients may be carried out, for example, by intraarterial, intravenous, or subcutaneous administration, as well as by intranasal, transbronchial, intramuscular, or oral methods well known to those skilled in the art, but are not limited thereto. Furthermore, administration may be systemic or local. If systemic side effects caused by drug administration become a problem, dosage can be minimized by local administration. Dosage varies depending on the tissue localization of the active ingredients of the pharmaceutical composition, the therapeutic objective, weight, age and symptoms of patients, method of administration, and such. However, one skilled in the art can appropriately select suitable dosages. The active ingredient should be administered in the range of typically 1 μg/kg to 50 mg/kg, preferably 10 μg/kg to 10 mg/kg, or more preferably 10 μg/kg to 5 mg/kg. The dose may be administered all at once, or several times over an appropriate interval.

If the compound can be encoded by a nucleic acid, such as DNA or RNA, gene therapy may be performed by incorporating this nucleic acid into a gene therapy vector. In particular, antisense polynucleotides of the PEG10 gene can be expressed by incorporation into gene therapy vectors, followed by administration to target cells. Vectors that may be used for such purposes include the viral vectors mentioned in the present invention. Dosage and administration methods vary with patient weight, age, and symptoms, however can be appropriately selected by those skilled in the art.

Individuals targeted for administration include, without limitation, mammals such as humans, monkeys, mice, rats, and rabbits, and other vertebrates. Application to non-human mammals such as mice, rats, and monkeys is useful in constructing a model for developing preventive or therapeutic methods for human diseases.

The pharmaceutical compositions of this invention are useful for prevention and therapy of diseases in which the PEG10 protein participates. Specifically, the pharmaceutical compositions of this invention may be used as preventive or therapeutic agents of diseases caused by PEG10 protein-mediated cell growth and/or apoptosis abnormality. In particular, the pharmaceutical compositions of this invention are useful for suppressing the growth of neoplasms such as tumors, and for inducing apoptosis. The pharmaceutical compositions of this invention are especially suitable for use in the prevention and therapy of HCCs.

Furthermore, the present invention provides methods of testing for and diagnosing hepatoma, wherein the methods comprise the step of investigating structural changes and aberrant expression of the PEG10 and/or SIAH gene. The present inventors studied PEG10 gene expression in 36 different HCCs, and surprisingly found that PEG10 gene expression was commonly enhanced in 35 of these. Since PEG10 gene expression was enhanced in the majority of HCCs, and extremely low in corresponding non-cancerous liver tissues, PEG10 may be an extremely useful diagnostic marker for hepatomas. SIAH1 expression is reduced in HCC cell lines with enhanced PEG10 gene expression, and apoptosis is induced upon the exogenous transfection of SIAH1 into HCC cells. This indicates that SIAH1 plays an important role in suppressing hepatocarcinogenesis. In addition, SIAH-1 was demonstrated to reduce PEG10 protein expression in a dose-dependent fashion. Accordingly, the imbalance between PEG10 and SIAH1 expression may be involved in hepatocarcinogenesis through apoptosis inhibition. Thus, the present inventors demonstrated for the first time that detecting aberrations in the PEG10 and/or SIAH gene enables reliable testing for hepatoma.

The testing and diagnosis of this invention can be carried out by detecting structural mutations or expression aberrations of the PEG10 and/or SIAH gene. For example, detection of structural variations in the PEG10 and/or SIAH gene can be performed by amplifying the PEG10 and/or SIAH gene from a subject's chromosomal DNA or mRNA, using PCR or the like, and then determining the nucleotide sequence. The presence or absence of a mutation, and the type of mutation, can be determined by comparison with the nucleotide sequence of the normal gene. In this invention, mutations include polymorphisms. That is, it is thought that by detecting single nucleotide polymorphisms (SNPs) in the PEG10 or SIAH gene, the prognosis for hepatic cancer, and the risk of its onset, can be tested and diagnosed. PEG10 gene mutation that elevates the activity of cell proliferation-enhancement or of cell death-suppression, and SIAH gene mutation that lowers the degradation activity of the PEG10 protein, are judged to be risk factors for the onset and progress of hepatic cancer. Also, to detect aberrations in the expression of the PEG10 and/or SIAH gene, for example, expression of the PEG10 gene or SIAH gene in patient-derived heptocytes can be analyzed by microarray analysis, quantitative or semi-quantitative RT-PCR, immunohistochemical staining, Western blotting, and such. Aberrations in expression are determined by comparison with expression in normal tissues. An aberration in expression includes an increase, decrease, or loss of expression. Increased PEG10 gene expression, or decreased SIAH gene expression, are judged to be risk factors for the onset and progress of hepatic cancer. The testing and diagnosis of this invention include risk testing performed prior to hepatic cancer onset, classification and malignancy evaluation performed after cancer onset, testing for the preparation of a therapeutic strategy, and so on. For example, after cancer onset, structural mutations or expression aberrations of the PEG10 and/or SIAH gene are detected by testing the tumor tissue, and determining the presence or absence of mutation, and the type of mutation and aberration. In addition, if testing prior to carcinogenesis reveals a high risk of hepatic carcinogenesis, the probability of detecting early-stage cancer can be elevated by setting a frequent preventive medical examination schedule. If detected after carcinogenesis, prognosis prediction or treatment selection can be appropriately carried out upon classifying or distinguishing the cancer. The testing of this invention can also be used to determine whether agents obtained by the screening of this invention are effective. This testing makes it possible to achieve tailor-made treatments to suit individual hepatic cancers.

In the above-mentioned testing of this invention, polynucleotides comprising a nucleotide sequence of the PEG10 or SIAH gene, or a portion of their complementary sequence, or antibodies against the PEG10 or SIAH protein, can be used. The polynucleotide comprising a nucleotide sequence of the PEG10 or SIAH gene, or a portion of their complementary strand, may be a polynucleotide comprising a nucleotide sequence of these genes, or a portion of their complementary strand, in mRNA, cDNA, or genomic DNA. Thus, for example, it may be a sequence in the protein-encoding region, the 5′ or 3′ UTS, an intron, exon, or sequence in the transcription regulatory region. The above-mentioned polynucleotide is preferably a polynucleotide comprising at least 15 or more continuous nucleotides within a nucleotide sequence of the PEG10 or SIAH gene, or complementary strand thereof, preferably 16 or more nucleotides, and more preferably 17, 18, or 20 or more nucleotides. These polynucleotides may be DNA or RNA. The above-mentioned polynucleotide may also be one that comprises modified nucleotides. Polynucleotides may be used in testing as probes, as immobilized polynucleotides in a DNA microarray, or as primers for RT-PCR and such. Also, the antibodies are useful for detecting PEG10 and/or SIAH gene expression at the protein level, and for performing tests through protein structure analyses. The present invention provides hepatic cancer tests or diagnostic reagents which include these polynucleotides or antibodies. The polynucleotides and antibodies may be bound to a carrier or to a support, for example, in the form of a DNA microarray and such. In addition, polynucleotides and antibodies may be appropriately combined with solvents and solutes. The tests and diagnostic reagents of this invention may be provided, for example, as aqueous solutions. The reagent of this invention may be a kit for testing and diagnosis, which is placed in an appropriate container and can include packaging and/or instructions. The container, packaging or instructions of a test or diagnostic reagent of this invention preferably describe the fact that the reagent can be used for testing and diagnosis of hepatic cancer, including HCCs, or describe other printed matter or a URL or the like that has this information (that the reagent can be used for testing and diagnosis of hepatic cancer, including HCCs), thereby linking the reagent to the information.

### BRIEF DESCRIPTION OF THE DRAWINGS

FIG. 1 features a photograph and an illustration showing PEG10 tissue expression and structure. A: Multiple-tissue Northern blot analysis of PEG10 in adult human tissues. B: PEG10 protein motifs predicted by the Simple Modular Architecture Research Tool (SMART ver.3).

FIG. 2 features photographs showing PEG10 protein expression in hepatoma cell lines and a primary HCC. A: PEG10 gene and protein expression in six hepatoma cell lines. RT-PCR was carried out using a PEG10-specific PCR primer set (upper panel). GAPDH served as an internal control. Immunoblotting was performed using anti-PEG10 antibody (bottom panel). The amount of protein applied in the SDS-PAGE was evaluated using Coomassie Brilliant Blue (CBB) staining. B: Subcellular PEG10 protein localization in HCC cell lines. (Magnification: ×600). C: Immunohistochemical staining of PEG10 in a primary HCC and in the corresponding non-cancerous liver tissue. (Magnification: ×600).

FIG. 3 features photographs and graphs showing the growth-promoting effect of PEG10 in human hepatoma cell lines. A: Colony formation by SNU423 and SNU475 cells after PEG10 gene transfer. The number of colonies transfected with plasmids expressing PEG10 relative to the mock vector was calculated (mean±SD). B: Expression of PEG10 protein in stable-transfectant (SNU423-PEG10) cells. C: Immunohistochemical staining of PEG10 in SNU423-PEG10 cells. (Magnification, ×1000).

FIG. 4 features graphs showing the growth-promoting effect of PEG10 in human hepatoma cell lines. A: The growth curve of SNU423-PEG10 cells cultured in RPM11640 containing 10% FBS. B: The growth curve of SNU423-PEG10 cells under serum-starved conditions (cultured in RPM11640 containing 0.1% FBS). C: Graphs of the cell cycles of mock and SNU423-PEG10 cells, cultured in RPMI1640 containing 0.1% FBS. Open columns: G0/G1 phase; shaded columns: S phase; and black columns: G2/M phase.

FIG. 5 features photographs and illustrations showing the interaction between PEG10 and SIAH-1/2 proteins. A: A yeast two-hybrid experiment. pAS2-1, or pAS2-1 containing PEG10 gene, was co-transfected into yeast strain AH109 with library vectors containing SIAH1 or SIAH2. B: An in vitro protein-binding assay for PEG10 and SIAH-1. Proteins from cells expressing PEG10 were incubated with resin that was either conjugated or not conjugated to SIAH-1. Eluted proteins were analyzed by immunoblotting using anti-His and anti-PEG10 antibodies. In the last two lanes, his-tagged SIAH-1 and cell lysate containing PEG10 were loaded directly on to the gel as controls. C: An in vitro protein-binding assay for PEG10 and SIAH-2. Proteins from cells expressing flag-tagged SIAH-2 were incubated with GST-PEG10 or GST, and purified with Glutathione sepharose beads. In the right three lanes, GST-PEG10 fusion protein, GST, and cell lysate containing flag-tagged SIAH-2 protein were loaded as controls.

FIG. 6 features photographs and graphs showing the effects of SIAH1 gene transfer on the viability of hepatoma cells, and on PEG10 expression. A: Expression of SIAH1 in hepatoma cell lines examined using RT-PCR. mRNAs extracted from normal liver tissue were used for comparison. B: Exogenous expression of myc-tagged SIAH-1 protein in SNU423 cells, caused by adenovirus-mediated SIAH1 gene transfer (MOI=100). C: Microscopic observation of five human hepatoma cell lines 48 hours after Ad-LacZ or Ad-SIAH1 infection at MOI=100 (Magnification, ×200). D: Ad-SIAH-induced Huh7 cell apoptosis (MOI=100); the Sub-G1 population of cells infected with Ad-SIAH1 or Ad-LacZ, examined 48 hours after FACS infection (left panels). Apoptotic cells were detected in green or yellow using TUNEL assays (right panels).

FIG. 7 features photographs and graphs showing the effects of SIAH1 gene transfer on the viability of hepatoma cells, and on PEG10 expression. A: Assessment of cell viability by MTT assay after Ad-SIAH1 infection at MOI=20, 50 and 100. B: Dose-dependent decrease of PEG10 protein after Ad-SIAH1 infection of HepG2 cells at different MOIs. Proteins were extracted 48 hours after infection, and analyzed by immunoblotting using anti-PEG10 antibody. C: The effect of PEG10 expression on SIAH-1-induced cell death. Cell viability was examined by MTT assay 48 hours after Ad-SIAH1 infection of PEG10-expressing cells (SNU423-PEG10), parental (SNU423) cells, or control (SNU423-Mock) cells. Data (mean±SD) represents the MTT activity of cells infected with Ad-SIAH1, relative to those infected with Ad-LacZ.

FIG. 8 features photographs and graphs showing growth suppression by antisense S-oligonucleotides designed to suppress PEG10. A: PEG10 protein levels in Huh7 cells transfected with either sense or antisense S-oligonucleotides. B: Colonies of Alexander and Huh7 cells after transfection with the sense or anti-sense S-oligonucleotides. The number of colonies of cells transfected with anti-sense S-oligonucleotides relative to sense S-oligonucleotides was examined (mean±SD).

### DETAILED DESCRIPTION

Herein below, the present invention will be specifically described using examples, however, it is not to be construed as being limited thereto. All references cited herein are incorporated as a part of the present invention.

### EXAMPLE 1

Cell Lines and Tissue Samples

Human embryonic kidney 293 cells (HEK293) and human hepatoma cell lines HepG2, Huh7 and Alexander were obtained from the American Type Culture Collection (ATCC, Rockville, MD). SNU423, SNU449, and SNU475 were obtained from the Korea cell-line bank. All cell lines were grown in monolayers in appropriate media supplemented with 10% fetal bovine serum and 1% antibiotic/antimycotic solution (Sigma, St. Louis, Mo.), and maintained at 37° C. in air containing 5% CO2. All HCCs and corresponding non-cancerous liver tissues were obtained with informed consent from patients who had undergone hepatectomy.

### EXAMPLE 2

Identification of a Novel Gene Frequently Up-Regulated in Hccs

Using a 23,040 gene, genome-wide cDNA microarray, the present inventors identified one commonly up-regulated EST from a total of 20 hepatitis B virus-positive or hepatitis C virus-positive HCCs (Okabe, H. et al., Cancer Res. 61, 2129-2137 (2001)). TaqMan PCR was used to confirm elevated expression of the gene (which corresponded to KIAA1051 (Hs. 137476)) in some tumors (data not shown). The relative expression ratios confirmed by TaqMan PCR showed a good correlation with those achieved using the cDNA micro array.

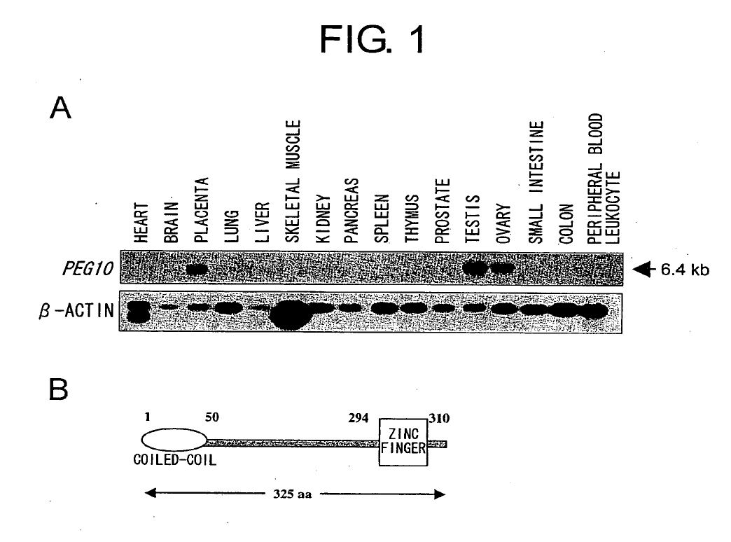

Multiple-tissue Northern blot analyses were carried out using cDNAs as probes. In these Northern blot analyses, human multiple-tissue blots (Clontech, Palo Alto, Calif.) were hybridized with a 32P-labeled PEG10 cDNA. Pre-hybridization, hybridization and washing were performed according to the supplier's recommendations. The blots were auto-radiographed with intensifying screens at −80° C. for 24 hours. These results showed that a 6.4-kb transcript was predominantly expressed in the placenta, testes and ovary (FIG. 1A). cDNA sequences covering almost the entire gene transcript were obtained using 5′ Rapid Amplification of cDNA ends (the 5′ RACE method), and the gene was eventually revealed to be identical to PEG10 (Ono, R. et al., Genomics 73, 232-237 (2001)). Simple Modular Architecture Research Tool (SMART ver.3, http://smart.embl-heidelberg.de) suggested that the predicted protein contained a coiled-coil motif (codons 1 to 50) as well as a zinc-finger motif (codons 294 to 310) (FIG. 1B).

### EXAMPLE 3

Expression of PEG10 in HCC Cell Lines and Primary HCCs

To investigate the role of PEG10 in HCCs, the present inventors generated a rabbit polyclonal antibody to the gene product. The polyclonal antibody to PEG10 was purified from the sera of immunized rabbits, using recombinant GST-PEG10 protein produced in E. coli.

Immunoblotting was carried out as follows: Cell extracts were prepared using lysis buffer (150 mM NaCl, 1% Triton X-100, 50 mM Tris-HCl (pH 7.4), and 1 mM DTT, with complete Protease Inhibitor Cocktail (Boehringer Mannheim, Mannheim, Germany). Proteins were separated using 10% SDS-PAGE and immunoblotted with the rabbit anti-PEG10 antibody. HRP-conjugated goat anti-rabbit IgG (Santa Cruz Biotechnology, Santa Cruz, Calif.) served as the secondary antibody, and signals were detected using the ECL Detection System (Amersham Pharmacia Biotech, Piscataway, N.J.).

Immunohistochemical staining was carried out as follows: Cultured cells on chamber slides were fixed with PBS containing 4% paraformaldehyde for 15 minutes, then rendered permeable with PBS containing 0.1% Triton X-100 for 2.5 minutes at room temperature. Frozen sections from primary HCCs and non-cancerous liver tissue were fixed with acetone for 15 minutes. The cells were incubated with 2% BSA in PBS for 24 hours at 4° C., and hybridized with the anti-PEG10 antibody. Antibodies were stained with fluorescent substrate-conjugated anti-rabbit secondary antibody (ICN Pharmaceuticals, Costa Mesa, Calif.), nuclei were then counter-stained with 4′, 6′-diamidine-2′-phenylindole dihydrochloride (DAPI), and fluorescent images were obtained using an ECLIPSE E800 microscope (Nikon, Tokyo, Japan).

RT-PCR analysis was carried out as follows: RT-PCR experiments were carried out in 20 μl volumes of PCR buffer (TaKaRa, Tokyo, Japan). Samples were denatured for four minutes at 94° C., followed by 20 (for GAPD) or 30 (for PEG10 and SIAH1) cycles at 94° C. for 30 seconds, 56° C. for 30 seconds, and 72° C. for 30 seconds. The GeneAmp PCR system 9700 (Perkin-Elmer, Foster City, Calif.) was used, and the primer sequences were as follows:

GAPDH (forward 5′-ACAACAGCCTCAAGATCATCAG-3′
(SEQ ID NO: 1)
and
reverse 5′-GGTCCACCACTGACACGTTG-3′
(SEQ ID NO: 2));
PEG10 (forward 5′-AACAACAACAACAACTCCAAGC-3′
(SEQ ID NO: 3)
and
reverse 5′-TCTGCACCTGGCTCTGCAC-3′ (SEQ ID NO: 4));
and
SIAH1 (forward 5′-TCCAACAATGACTTGGCGAGT-3′
(SEQ ID NO: 5)
and
reverse 5′-CTTTTTCTGTGTGTGGCAGAG-3′
(SEQ ID NO: 6)).

(See Example 6)

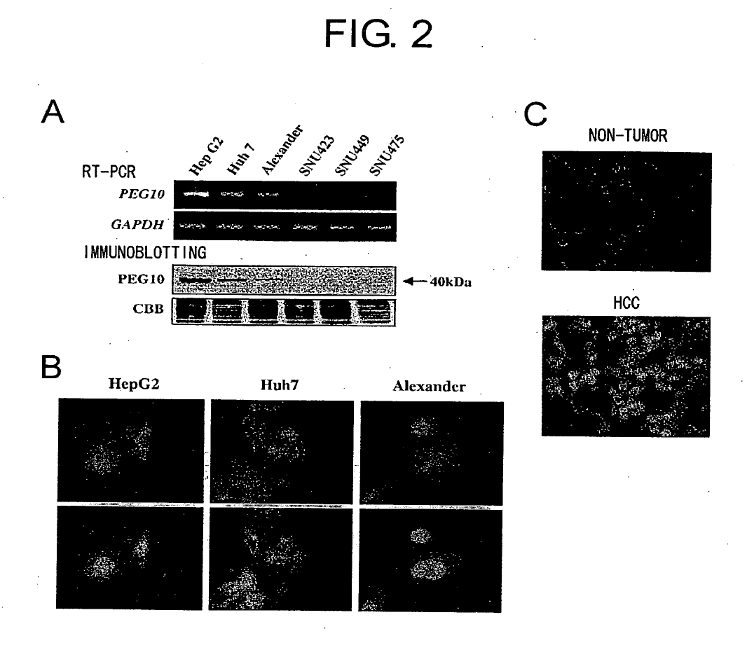

HepG2, Huh7 and Alexander cells constantly expressed the 40-kD PEG10 protein (FIG. 2A), and immunohistochemical staining revealed that PEG10 was located in both the nucleus and cytoplasm of those cells (FIG. 2B). Staining was not evident in SNU423, SNU449 and SNU475 cells, consistent with the results of Western analysis. Strong nuclear and cytoplasmic staining of PEG10 in the tumor tissues was detected in 15 of the 16 primary HCCs that differed from the 20 HCCs used for cDNA microarray analysis. However, this staining was not detected in the corresponding normal tissues (FIG. 2C).

### EXAMPLE 4

PEG10 Promotion of the Growth of Human Hepatoma Cells

To analyze the effects of PEG10 gene transfer on the growth of hepatoma cells, the present inventors transfected an expression plasmid comprising PEG10 into two cell lines (SNU423 and SNU475), which had shown no endogenous PEG10 protein expression. They then carried out colony-formation assays and proliferation-suppressing assays. Specifically, cells were transfected with plasmid vector expressing the entire coding region of PEG10, using FuGENE6 reagent according to the supplier's protocol (Boehringer). The cells were cultured with an appropriate concentration of geneticin for two weeks, fixed with 100% methanol, and then stained using Giemsa solution. Colonies larger than 1 mm were counted two weeks after transfection with pcDNA 3.1(+) (empty vector), pcDNA 3.1(−)/PEG10 (PEG10 anti-sense strand transcription), or pcDNA 3.1(+)/PEG10 (PEG10 sense strand transcription). Cells transfected with sense (5′-CCTCGCGTGGTGAGTA-3′ (SEQ ID NO: 7)) or anti-sense (5′-TACTCACCACGCGAGG-3′ (SEQ ID NO: 8)) PEG10 S-oligonucleotides were stained in the same manner (Example 7).

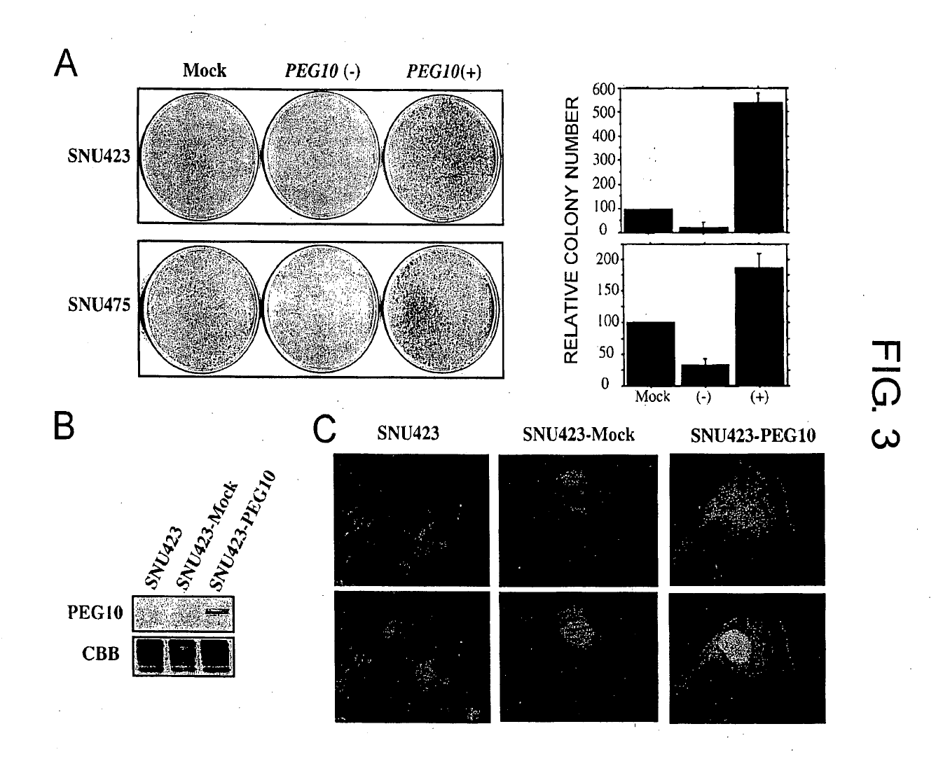

Compared to mock-transfected or anti-sense plasmid-transfected clones, the PEG10 sense plasmid vector promoted colony formation in both cell lines (FIG. 3A). PEG10 protein expression in the sense vector-transfected cell lines was confirmed using immunoblotting. The results of colony-formation assays were confirmed by three independent experiments.

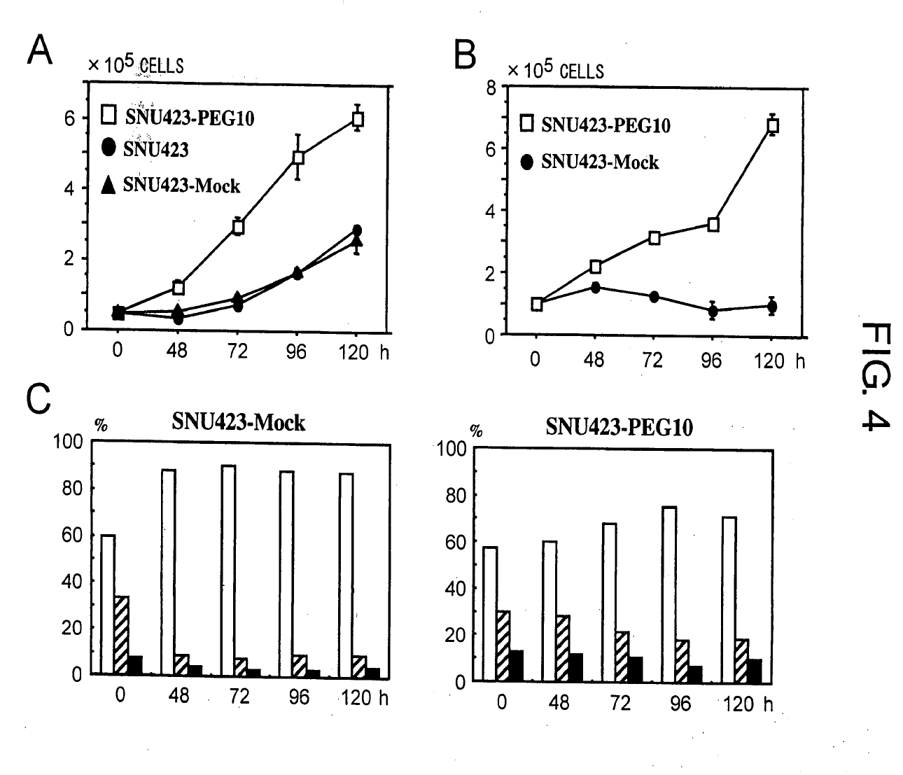

To further investigate the growth-promoting effects of PEG10 the present invention generated stable transfectants using SNU423 cells in which endogenous PEG10 expression was absent. Cell cycles were analyzed using flow cytometry. Specifically, 1×105 cells were collected by trypsinization at given times, and fixed in 70% cold ethanol. Cells were treated with RNase and propidium iodide (50 μg/ml) in PBS, and analyzed using a FACScan (Becton Dickinson, San Jose, Calif.). The PEG10-stable transfectants showed continual PEG10 protein expression, mainly in the nucleus (FIGS. 3B and 3C), and were revealed to have significant growth promotion compared to parent or mock cells (FIG. 4A). Under conditions of serum starvation (0.1% FBS), mock cells rapidly went into growth arrest, but cells stably expressing PEG10 continued to proliferate (FIG. 4B). Cell-cycle analysis indicated arrest of mock cells at the G1 phase, while PEG10-transfected cells continued to proliferate. The cycle of the PEG10-transfected cell population was not affected by serum starvation (FIG. 4C).

### EXAMPLE 5

Interaction of PEG10 with SIAH-1 and SIAH-2

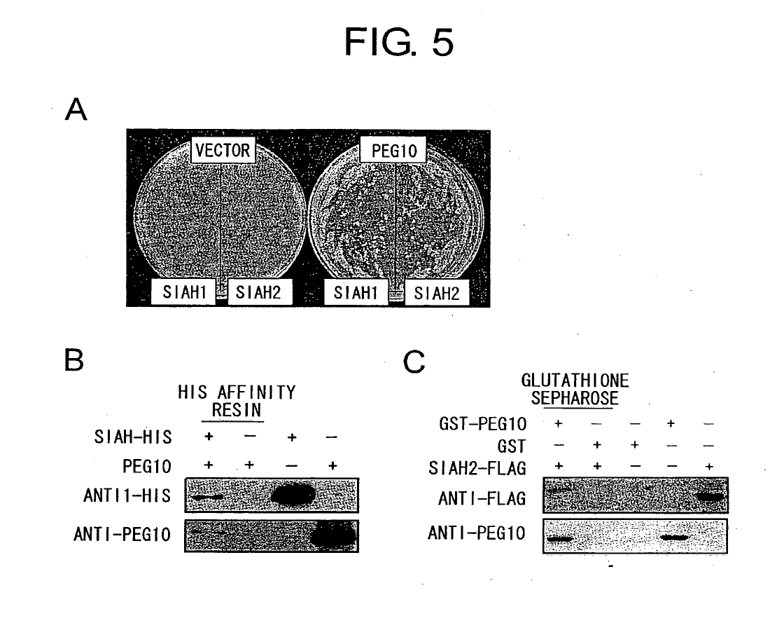

To examine the oncogenic mechanism of PEG10, the present inventors searched for PEG10-interacting proteins using a yeast two-hybrid screening system. The yeast two-hybrid assay was carried out with the MATCHMAKER GAL4 Two-Hybrid System 3, according to the manufacturer's protocol (Clontech, Palo Alto, Calif.). The present inventors cloned the entire PEG10-encoding sequence into the EcoR I-Sal I site of the pAS2-1 vector, and used this as a bait to screen a human testis cDNA library (Clontech). Of the clones identified, those homologous to Drosophila seven in absentia (SIAH1 and SIAH2) interacted with PEG10 by simultaneous transformation with pAS2.1-PEG10 and pACT2-SIAH1 or pACT2-SIAH2 (FIG. 5A). To confirm the interaction of PEG10 with SIAH-1, the present inventors prepared recombinant His-tagged SIAH-1 protein, and detected this association when the PEG10 protein was expressed in mammalian cells (FIG. 5B). In addition, the present inventors demonstrated the association of GST-PEG10 fusion protein with flag-tagged SIAH-2 protein, expressed in HEK293 cells (FIG. 5C).

The in vitro protein-binding assay was carried out as follows: The entire coding regions of SIAH1 and SIAH2 were amplified using the primers 5′-CGCGAATTCCGCCCACAGAAATGAGCC-3′ (SEQ ID NO: 9) and 5′-CATCTCGAGACATGGAAATAGTTACATTGATGC-3′ (SEQ ID NO: 10), or 5′-TGCGAATTCCATGGTTGGTTCGGAGC-3′ (SEQ ID NO: 11) and 5′-GTGCTCGAGGACAACATGTAGAAATAGTAAC-3′ (SEQ ID NO: 12) respectively, and cloned into appropriate cloning sites of the pET21b vector (Novagen, Madison, Wis.) or pCMV-Flag5 (Sigma). Recombinant His-tagged SIAH-1 protein was prepared using the XpressTM system (Invitrogen, Carlsbad, Calif.) according to the manufacturer's recommendations. ProBondTM histidine affinity resin (Invitrogen) was incubated with or without 10 μg of His-tagged SIAH-1 protein at 4° C. for one hour, and then washed well with a binding buffer (20 mM NaH2PO4, 500 mM NaCl, pH 7.8). This resin was then incubated in NP-40 lysis buffer (150 mM NaCl, 50 mM Tris (pH8.0), and 1% NP-40) with 50 μg of cell lysate from SNU423-PEG10 cells exogenously over-expressing PEG10. The resin was twice washed with wash buffer (20 mM NaH2PO4 and 500 mM NaCl) at each of pH 7.8, pH 6.0, and pH 5.5, and proteins were then eluted using an elution buffer (300 mM imidazole in wash buffer). The eluted proteins were analyzed by immunoblotting using anti-His probe antibody (Santa Cruz) or anti-PEG10 antibody. Similarly, GST or GST-PEG10 fusion protein was immobilized on Glutathione SepharoseTM 4B beads (Amersham Pharmacia Biotech, Uppsala, Sweden), and incubated with lysates from HEK293-SIAH2 cells over-expressing Flag-tagged SIAH2. Bound proteins were eluted with elution buffer (120 mM NaCl, 50 mM Tris-HCl (pH 8.0), and 20 mM glutathione (Sigma)), and analyzed by immunoblotting using anti-Flag and anti-PEG10 antibodies.

### EXAMPLE 6

Induced HCC Cell Death and Reduced Expression of PEG10 Protein in Response to SIAH1 Gene Transfer

SIAH-1 participates in E2-dependent ubiquitination of target proteins through its RING finger domain, and induces apoptosis in several cell lines. Hence, the present inventors hypothesized that SIAH-1 might also degrade PEG10 via the ubiquitin-proteasome pathway. To examine this hypothesis and analyze the function of SIAH-1 in HCC cells, the present inventors generated recombinant adenoviruses expressing myc-tagged SIAH-1 protein (Ad-SIAH1) and LacZ (Ad-LacZ). Generation and preparation of adenoviruses expressing SIAH1 was achieved using the Adenovirus Expression Vector Kit (TaKaRa) according to the supplier's protocol. First, the entire SIAH1 coding region was amplified and cloned into an appropriate site in the pcDNA3.1/myc-C vector (Invitrogen). The myc-tagged SIAH1 fragment was then cloned into the cosmid vector pAxCAwt, which was supplied in the kit (TaKaRa).

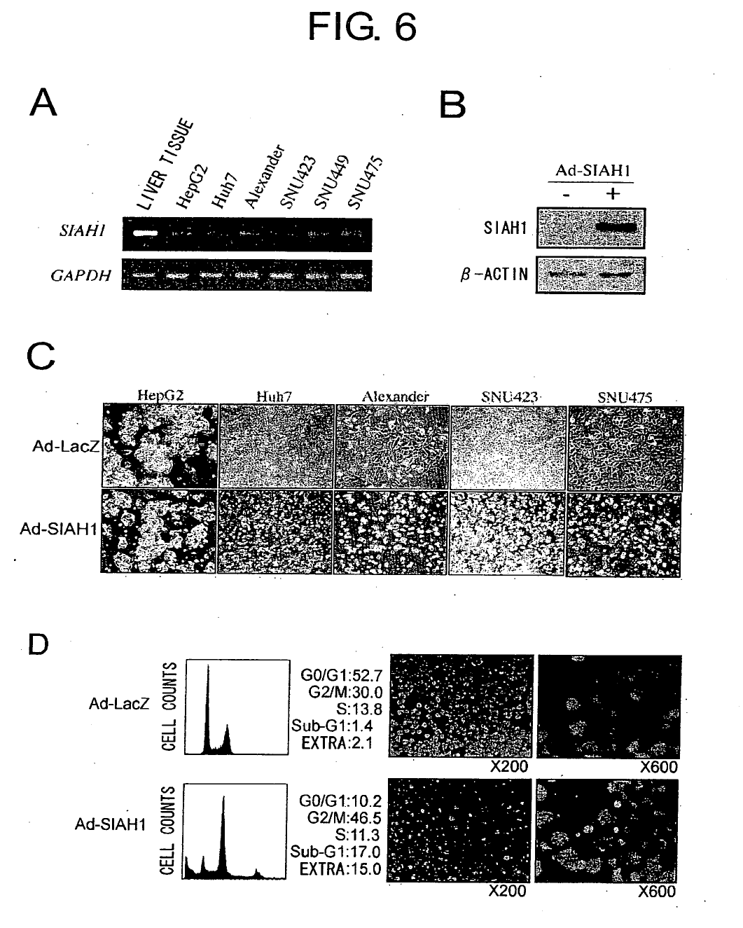

Semi-quantitative RT-PCR demonstrated that SIAH1 expression was decreased in all six hepatoma cell lines examined, compared to normal liver tissue (FIG. 6A). The present inventors infected five hepatoma cell lines (HepG2, Huh7, Alexander, SNU423 and SNU475) with Ad-SIAH1 or Ad-LacZ. The transfection efficiencies of each of these cells was revealed to be 67.3% to 100% at MOI=100. Immunoblotting analysis using anti-myc antibody confirmed exogenous expression of the myc-tagged SIAH-1 protein 24 hours after infection (FIG. 6B). In all five cell-lines, cell death in Ad-SIAH1-infected cultures increased significantly (FIG. 6C).

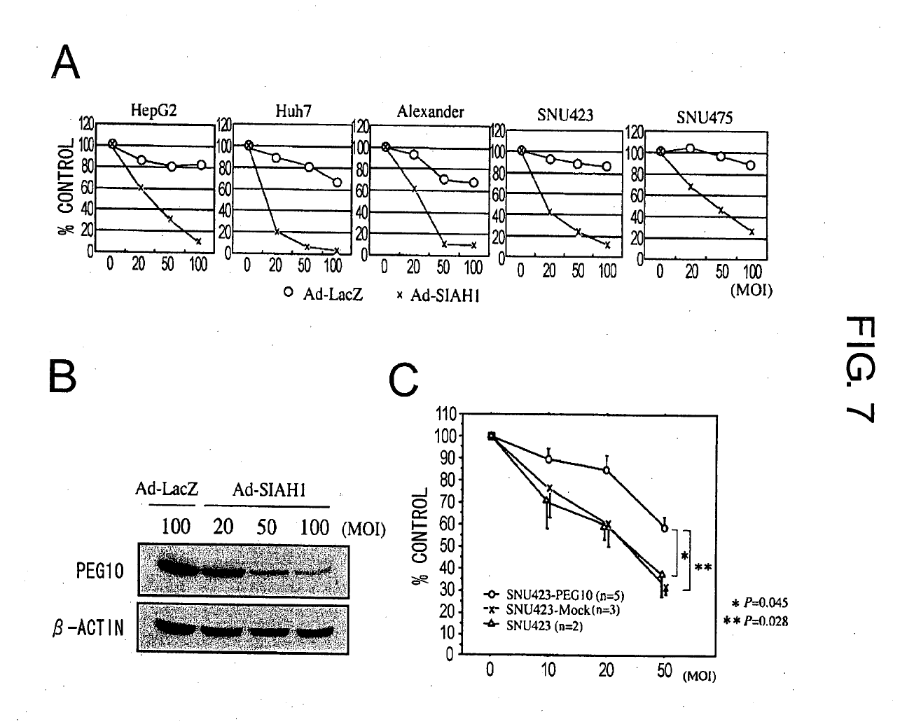

Flow-cytometry demonstrated that induction of SIAH-1 expression significantly increased the numbers of cells in G2/M and sub-G1 populations. TUNEL assays demonstrated that the number of apoptotic cells infected with Ad-SIAH1 was significantly greater than for cells infected with Ad-LacZ (FIG. 6D). A 3-(4,5-dimethylthiazol-2-yl)-2,5-diphenyltetrazolium bromide (MTT) assay was carried out as follows: Cells were plated in 6-well plates at densities of 1×105 cells/well, and infected with Ad-SIAH1 or Ad-LacZ at MOI=0, 20, 50, or 100. After 72 hours of infection, MTT assays were carried out as mentioned previously (Akashi, H. et al., Int. J. Cancer 88, 873-880 (2000)). Cell viability at each MOI was represented by absorbance compared to that of the control (MOI=0). Notably, after 72 hours of infection, the MTT assay demonstrated a dose-dependent decrease in viability for cells infected with Ad-SIAH1 (FIG. 7A). Immunoblotting was used to examine PEG10 protein levels 48 hours after infection. In three of the HCC cell lines examined (HepG2, Huh7 and Alexander; FIG. 7B), a reduced response to Ad-SIAH1 was documented. This was not observed for Ad-LacZ. To determine whether PEG10 protein over-expression could protect hepatocytes from SIAH-1-induced cell death, SNU423-PEG10 cells stably expressing exogenous PEG10 were constructed, and then infected with Ad-SIAH1. The viability of SNU423-PEG10 cells 48 hours after Ad-SIAH1 infection was significantly greater than that of parent SNU423 cells or control SNU423-Mock cells (FIG. 7C). This indicates that PEG10 comprises the activity of protecting against SIAH1-mediated cell death.

### EXAMPLE 7

Suppression of Hepatoma Cell Growth by PEG10 Anti-Sense Oligonucleotides

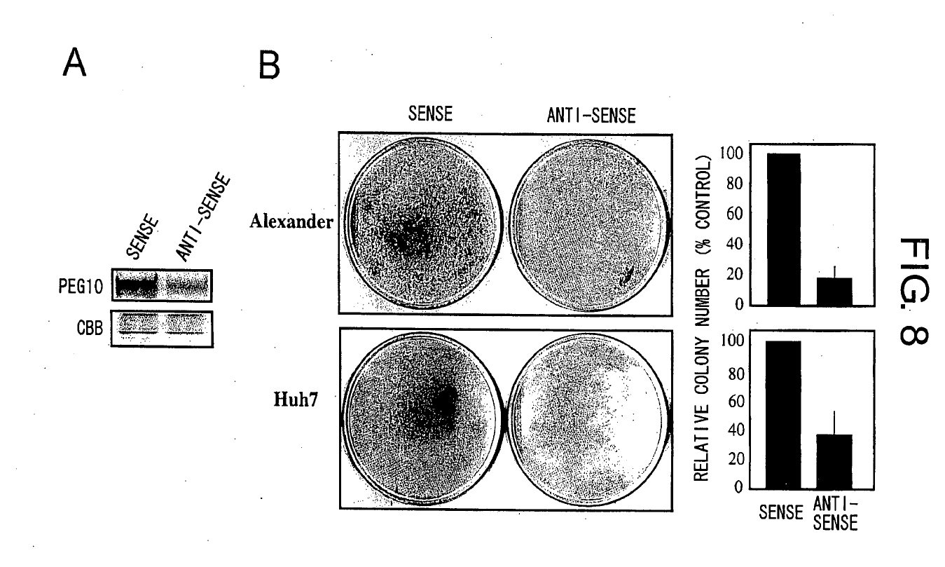

To examine whether PEG10 suppression would retard growth and/or induce the death of HCC cells, the present inventors designed various anti-sense S-oligonucleotides (Example 4). Of these, anti-sense S-oligonucleotides that encompassed the first exon-intron boundary (SEQ ID NO: 8) significantly decreased endogenous expression of PEG10 in Alexander and Huh7 cells constantly expressing large quantities of PEG10 (FIG. 8A). The other investigated anti-sense or control S-oligonucleotides did not have this reducing effect (FIG. 8A). Anti-sense S-oligonucleotide transfection significantly reduced colony formation in these two cell lines (FIG. 8B). However, growth-suppressive effect was not observed when anti-sense S-oligonucleotides were introduced into SNU423 cells which did not express endogenous PEG10 (data not shown). The colony-formation assay results were confirmed in three independent experiments.

### INDUSTRIAL APPLICABILITY

The present invention provides novel methods for the regulation of cell growth and cell death using PEG10. Furthermore, the present invention enables screening for cancer inhibitors which target PEG10. PEG10 protein expression is enhanced in most hepatocellular carcinomas, but is hardly observed in normal liver. This characteristic indicates that PEG10 is an ideal target molecule for the prevention or treatment of hepatocellular carcinomas. The present invention contributes greatly to the development of novel therapeutic strategies for hepatocellular carcinomas.

What is claimed is:

1. A method of promoting or suppressing cell growth, wherein the method comprises the step of increasing or decreasing PEG10 protein level in a cell, respectively.

2. A method of suppressing or promoting cell death, wherein the method comprises the step of increasing or decreasing PEG10 protein level in a cell, respectively.

3. The method of claim 1 or 2, wherein the step of increasing or decreasing PEG10 protein level in a cell is the step of suppressing or promoting the interaction level of the PEG10 protein with a SIAH protein in a cell.

4. The method of claim 1 or 2, wherein the step of increasing PEG10 protein level in a cell is the step of exogenously expressing the PEG10 gene.

5. The method of claim 1 or 2, wherein the step of decreasing PEG10 protein level in a cell is the step of transferring antisense polynucleotides against the PEG10 gene into a cell, or expressing the polynucleotides in a cell.

6. The method of claim 1 or 2, wherein the cell is a cancer cell.

7. The method of claim 6, wherein the cancer cell is a hepatoma cell.

8. The method of claim 7, wherein the hepatoma cell is a hepatocellular carcinoma cell.

9. A method of screening for compounds comprising the activity of regulating cell growth or cell death, the method comprising the steps of:
(a) detecting PEG10 gene expression level in a cell in the presence of a sample comprising a test compound; and
(b) selecting compounds comprising the activity of increasing or decreasing the expression level.

10. A method of screening for compounds comprising the activity of regulating cell growth or cell death, the method comprising the steps of:
(a) detecting the interaction between the PEG10 protein and a SIAH protein in the presence of a sample comprising a test compound; and
(b) selecting compounds comprising the activity of increasing or decreasing the interaction.

11. A method of screening for compounds comprising the activity of regulating cell growth or cell death, the method comprising the steps of:
(a) culturing cells exogenously expressing the PEG10 protein in the presence of a sample comprising a test compound; and
(b) selecting compounds comprising the activity of increasing or decreasing growth or cell death of the cells.

12. A pharmaceutical composition for preventing or treating cell proliferative diseases, comprising compounds able to be isolated by a method of any one of claims 9 to 11.

13. The pharmaceutical composition of claim 12, comprising an antisense polynucleotide against the PEG10 gene.

14. A method of testing or diagnosing hepatoma, wherein the method comprises the step of detecting structural changes or aberrant expression of the PEG10 gene and/or SIAH gene.

15. The method of testing or diagnosis of claim 14, wherein the hepatoma is a hepatocellular carcinoma.

16. A reagent for testing or diagnosing hepatoma, comprising a polynucleotide which comprises a portion of the PEG10 gene or SIAH gene nucleotide sequence, or a portion of a complementary strand thereof, or an antibody that binds to the PEG10 protein or SIAH protein.

17. The reagent for testing or diagnosing hepatoma of claim 16, wherein the hepatoma is a hepatocellular carcinoma.


SEQUENCE LISTING (12 sequences)

SEQ ID NO: 1 - DNA, 22 bp, Artificial Sequence
  Description of Artificial Sequence artificially synthesized sequence
acaacagcctcaagatcatcag

SEQ ID NO: 2 - DNA, 20 bp, Artificial Sequence
  Description of Artificial Sequence artificially synthesized sequence
ggtccaccactgacacgttg

SEQ ID NO: 3 - DNA, 22 bp, Artificial Sequence
  Description of Artificial Sequence artificially synthesized sequence
aacaacaacaacaactccaagc

SEQ ID NO: 4 - DNA, 19 bp, Artificial Sequence
  Description of Artificial Sequence artificially synthesized sequence
tctgcacctggctctgcac

SEQ ID NO: 5 - DNA, 21 bp, Artificial Sequence
  Description of Artificial Sequence artificially synthesized sequence
tccaacaatgacttggcgagt

SEQ ID NO: 6 - DNA, 21 bp, Artificial Sequence
  Description of Artificial Sequence artificially synthesized sequence
ctttttctgtgtgtggcagag

SEQ ID NO: 7 - DNA, 16 bp, Artificial Sequence
  Description of Artificial Sequence artificially synthesized sequence
cctcgcgtggtgagta

SEQ ID NO: 8 - DNA, 16 bp, Artificial Sequence
  Description of Artificial Sequence artificially synthesized sequence
tactcaccacgcgagg

SEQ ID NO: 9 - DNA, 27 bp, Artificial Sequence
  Description of Artificial Sequence artificially synthesized sequence
cgcgaattccgcccacagaaatgagcc

SEQ ID NO: 10 - DNA, 33 bp, Artificial Sequence
  Description of Artificial Sequence artificially synthesized sequence
catctcgagacatggaaatagttacattgatgc

SEQ ID NO: 11 - DNA, 26 bp, Artificial Sequence
  Description of Artificial Sequence artificially synthesized sequence
tgcgaattccatggttggttcggagc

SEQ ID NO: 12 - DNA, 31 bp, Artificial Sequence
  Description of Artificial Sequence artificially synthesized sequence
gtgctcgaggacaacatgtagaaatagtaac